(19) United States
(12) Patent Application Publication
(10) Pub. No.: US 2018/0200526 A1
(43) Pub. Date: Jul. 19, 2018

(54) IMPLANTABLE ANTENNA FOR PHYSIOLOGICAL MONITORING OR STIMULATION OF TISSUE

(71) Applicant: University of Southern California, Los Angeles, CA (US)

(72) Inventors: Konstantina S. Nikita, Los Angeles, CA (US); Asimina Kiourti, Columbus, OH (US)

(21) Appl. No.: 15/872,876

(22) Filed: Jan. 16, 2018

Related U.S. Application Data
(60) Provisional application No. 62/448,277, filed on Jan. 19, 2017.

(51) Int. Cl. A61N 1/372; A61B 5/00

(52) U.S. Cl. CPC A61N 1/37229; A61N 1/37252; A61B 5/0031

(57) ABSTRACT

An antenna for use in living tissue and designed to at least one of receive or transmit signals includes a conductive ground plane. The antenna further includes a first conductive patch having at least a first slot. The antenna further includes a second conductive patch having at least a second slot, the conductive ground plane, the first conductive patch, and the second conductive patch being stacked. The antenna further includes a first dielectric substrate layer located between the conductive ground plane and the first conductive patch. The antenna further includes a second dielectric substrate layer located between the first conductive patch and the second conductive patch.

### CROSS REFERENCE TO RELATED APPLICATIONS

This application claims the benefit and priority of U.S. Provisional Application No. 62/448,277, entitled “IMPLANTABLE ANTENNA FOR PHYSIOLOGICAL MONITORING OR STIMULATION OF TISSUE,” filed on Jan. 19, 2017, the entire disclosure of which is hereby incorporated by reference herein in its entirety.

### BACKGROUND

### 1. Field

The present disclosure is directed to implantable or ingestible antennas designed for use in living tissue and designed to transmit and receive data regarding a connected sensor or stimulator also located within the living tissue, and methods for optimizing performance of the antennas.

### 2. Description of the Related Art

Implantable antennas may be integral components of many different types of implanted devices, such as devices that operate inside the human body and enable sensing and/or stimulating of living tissue (e.g., cardiac pacemakers, intra-cranial pressure monitors, deep brain neural sensors and modulators, and the like). Specifically, implantable antennas enable single or bi-directional wireless communication between the associated implanted device and exterior monitoring and/or control equipment (i.e., a remote device).

Design of such implantable antennas includes consideration of many variables, including the dielectric loading of the environment in which they are intended to operate. Such dielectric loading includes the dielectric loading of the surrounding biological tissues along with exterior air. In order to take such consideration of the dielectric loading, implantable antenna design may be performed inside realistically shaped and sized anatomical models of the human body. However, this approach is very time-consuming, as simulation software requires extensive amounts of time to solve the related geometries. In other cases, implantable antenna design may be performed inside canonically-shaped (e.g., cube or rectangular) models of the human body. These models are again relatively large in size, requiring extensive amounts of time to perform the simulations. Smaller canonically-shaped models have also been pursued in the past. Nevertheless, to date, there have been no standardized methods to accelerate the design process and optimally select: a) shape, size, and overall geometry of the tissue model, b) antenna placement inside the tissue model, and c) antenna shape and parameters. Therefore, it is desirable to create an antenna and a method for optimizing the antenna that can be performed in a relatively quick manner.

### SUMMARY

Described herein is an antenna for use in living tissue and designed to at least one of receive or transmit signals. The antenna includes a conductive ground plane. The antenna further includes a first conductive patch having at least a first slot. The antenna further includes a second conductive patch having at least a second slot, the conductive ground plane, the first conductive patch, and the second conductive patch being stacked. The antenna further includes a first dielectric substrate layer located between the conductive ground plane and the first conductive patch. The antenna further includes a second dielectric substrate layer located between the first conductive patch and the second conductive patch.

Also disclosed is an implantable or ingestible device for physiological monitoring or stimulation of tissue. The implantable or ingestible device includes an antenna designed to be connected to a sensor or a stimulator for wirelessly sending and receiving monitoring or stimulation data of the tissue. The antenna includes a conductive ground plane. The antenna further includes at least two vertically stacked conductive patches including a lower conductive patch and an upper conductive patch, the at least two vertically stacked conductive patches configured to at least partially provide the monitoring or stimulation data to at least one of the sensor, the stimulator, or a remote device, and having one or more slots that assist in miniaturization. The antenna further includes one or more dielectric substrate layers that insulate the conductive ground plane from the lower conductive patch, the lower conductive patch from the upper conductive patch, and the upper conductive patch from the tissue.

Also disclosed is a method of optimizing antenna parameters of an implantable or ingestible antenna. The method includes randomly or manually initializing each antenna parameter of a plurality of antenna parameters. The method further includes manually updating at least some of the plurality of antenna parameters until a first desired goal corresponding to antenna behavior is obtained. The method further includes automatically updating, using an optimization routine, at least some of the plurality of antenna parameters a predetermined quantity of iterations. The method further includes selecting, based on optimized antenna behavior, a plurality of final antenna parameters.

### BRIEF DESCRIPTION OF THE DRAWINGS

FIG. 1 illustrates a system for sensing or stimulating living tissue that includes a sensor or stimulator along with an ingestible or implantable antenna according to an embodiment of the present invention;

FIG. 2 illustrates a cross-sectional view of the antenna of FIG. 1 according to an embodiment of the present invention;

FIG. 3 illustrates a top-down view of a conductive ground plane and two conductive patches of the antennas of FIG. 1 according to an embodiment of the present invention;

FIG. 4 illustrates an enlarged view of one of the conductive patches of FIG. 3 according to an embodiment of the present invention;

FIG. 5 is a flowchart illustrating a method for refining and optimizing antenna parameters of an antenna according to an embodiment of the present invention;

FIG. 6 is an example implementation of the method of FIG. 5 according to an embodiment of the present invention;

FIGS. 7A and 7B illustrate example tissue boxes used for antenna simulation and include a single layer tissue model (SLTM) and a multi-layer tissue model (MLTM) according to an embodiment of the present invention;

FIG. 8 is a graph illustrating a reflection coefficient at a desired frequency of an antenna created using the method of FIG. 5 and a SLTM tissue box for the simulations according to an embodiment of the present invention;

FIG. 9 is a graph illustrating a reflection coefficient at a desired frequency of an antenna created using the method of FIG. 5 and a MLTM tissue box for the simulations according to an embodiment of the present invention;

FIG. 10 is a chart illustrating optimized slot lengths of the antennas created using the method of FIG. 5 with the SLTM and the MLTM tissue boxes according to an embodiment of the present invention; and

FIG. 11 is a chart illustrating simulation time of simulations performed during optimization of the antennas created using the method of FIG. 5 with the SLTM and the MLTM tissue boxes according to an embodiment of the present invention.

### DETAILED DESCRIPTION

Disclosed herein are optimized antennas, along with methods for optimizing antennas. The antennas and methods for optimization provide several benefits and advantages. For example, the optimized antenna has a smaller size (radius and thickness) than conventional antennas. The optimized antenna further provides improved performance (as measured by reflection coefficient, antenna gain, and the like at a desired frequency) relative to conventional antennas. The methods for optimization allow the antenna to achieve these benefits. The methods further provides the advantage of relatively fast antenna optimization for any situation (i.e., for any frequency and implantation or ingestion location).

An exemplary antenna includes a conductive ground plane and two or more conductive patches vertically stacked with the conductive ground plane. Each of the two or more conductive patches includes at least one slot extending inward from a perimeter of the corresponding patch to create a serpentine path for current to flow along the patch. The antenna further includes dielectric substrate layers located between each of the conductive ground plane and the conductive patches. The antenna may also include a superstrate layer that covers or encapsulates some or all of the antenna and provides for biocompatibility with the corresponding tissue.

An exemplary method includes initializing antenna parameters (such as a quantity of conductive patches, a quantity of the slots, a slot length of the slots, or the like) either manually or automatically. The method further includes manually updating the antenna parameters until a desired goal (such as a desired reflection coefficient) is achieved. After refinement, the antenna parameters may be updated using an optimization routine to optimize performance of the antenna.

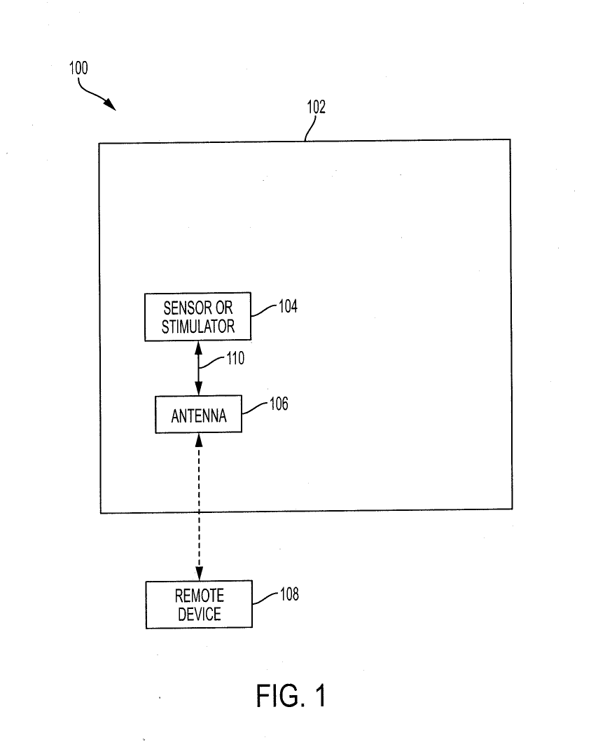

Turning to FIG. 1, a system 100 for stimulating or sensing data in living tissue 102 is shown. Living tissue refers to the fact that the tissue is organic; the system 100 may be used inside of organic tissue that is dead, which may still be considered living tissue due to the fact that the tissue at one point was alive. In some embodiments, the system 100 may be used in other dielectric materials such as soil, rubber, plastic, or the like.

The system 100 may include a sensor or stimulator 104, an antenna 106 electrically coupled to the sensor or stimulator 104, and a remote device 108 capable of wirelessly communicating with the antenna 106. The sensor or stimulator 104 may include a sensor that is designed to detect data corresponding to the living tissue 102. For example, the sensor 104 may include a voltage or current sensor for detecting a voltage or current flowing through the living tissue 102, a temperature sensor for detecting a temperature of the living tissue 102, or the like. The sensor or stimulator 104 may include a stimulator that is designed to stimulate the living tissue 102. For example, the stimulator 104 may be designed to periodically or continuously apply an electric charge to the living tissue 102. As an example, the living tissue 102 may include a brain or nerve, and the stimulator 104 may mask a signal in the brain or nerve.

The antenna 106 may be electrically coupled to the sensor or stimulator 104 via a physical connector 110. For example, the physical connector 110 may include a coaxial cable or connector, a twisted-pair cable, or the like. In that regard, the antenna 106 is implantable (including injectable via syringe) or ingestible (i.e., consumed or otherwise placed inside of the living tissue via an existing orifice in the tissue). In various embodiments, the antenna 106 may instead be designed for use inside dielectric materials other than living tissue, such as soil, rubbers, and the like.

In some embodiments, the antenna 106 may be mechanically connected to the sensor or stimulator 104. In some embodiments, the antenna 106 may be mechanically separated from the sensor or stimulator 104 and connected via a cable or wire. In either case, the combination of the antenna 106 and the sensor or stimulator 104 may be referred to as an implantable device.

The antenna 106 may be designed to at least one of transmit or receive data from the remote device 108. For example, the sensor 104 may detect data corresponding to the living tissue and the antenna 106 may transmit the detected data to the remote device 108. As another example, the remote device 108 may be designed to transmit a signal, such as a pulse train signal, to the antenna 106, and the antenna 106 may transmit a signal to the stimulator 104 such that the stimulator 104 may stimulate the living tissue 102 according to the signal.

The antenna 106 and/or the simulator or stimulator 104 may be implantable or ingestible. Implantation may include any type of implantation including surgical, injectable (i.e., the antenna 106 is implanted via injection), or the like. Ingesting may include swallowing or inserting the antenna 106 and/or the simulator or stimulator 104 into the living tissue via any existing orifice.

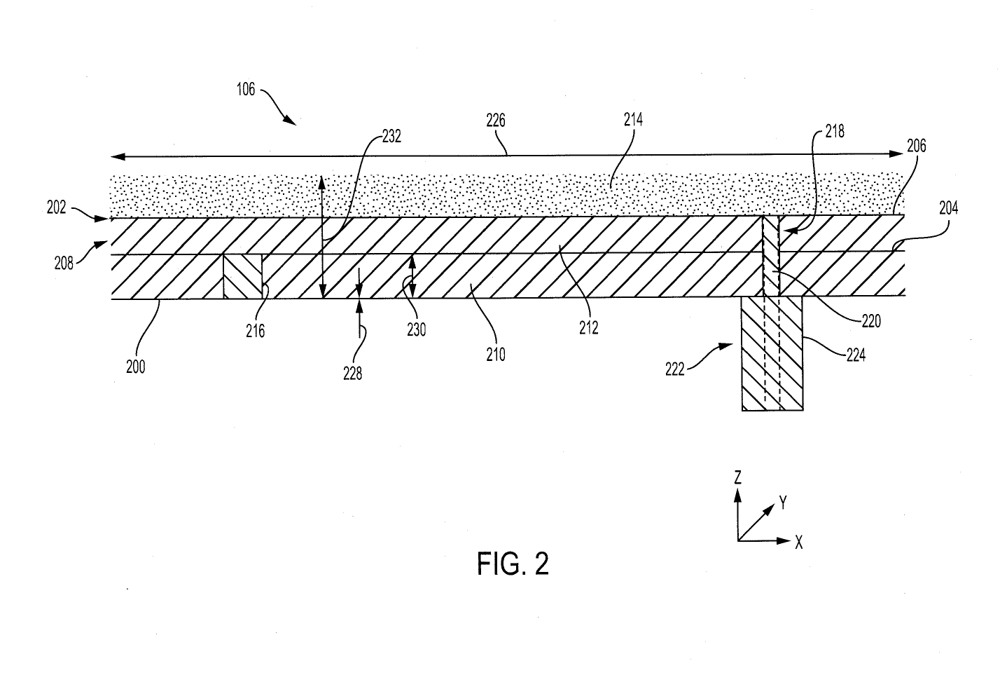

Turning now to FIG. 2, additional details of the antenna 106 are shown. The antenna 106 may include a conductive ground plane 200, at least two vertically stacked conductive patches 202 (including a first conductive patch 204 and a second conductive patch 206), and a plurality of dielectric substrate layers 208. Each of the conductive ground plane 200 and the at least two vertically stacked conductive patches 202 may include a conductive material, such as a metal. For example, the conductive ground plane 200 and the conductive patches 202 may include copper, gold, or the like.

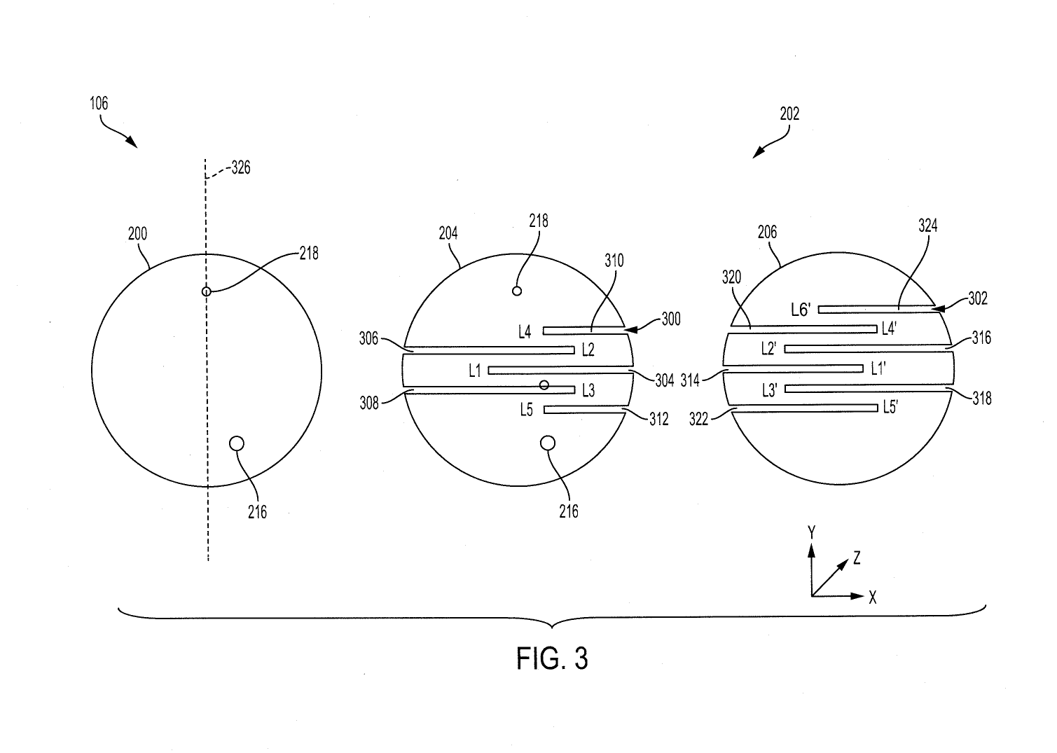

Referring now to FIGS. 2 and 3, the antenna 106 may have a shape that resembles a disk. In that regard, each of the conductive ground plane 200, the conductive patches 202, and the dielectric substrate layers 208 may be disk shaped and may be stacked above each other.

The dielectric substrate layers 208 may include a first dielectric substrate layer 210 located between the conductive ground plane 200 and the first conductive patch 204. The dielectric substrate layers 208 may further include a second dielectric substrate layer 212 located between the first conductive patch 204 and the second conductive patch 206. The dielectric substrate layers 208 may further include a dielectric superstrate layer 214 located above the second conductive patch 206 such that the second conductive patch 206 is located between the dielectric superstrate layer 214 and the second dielectric substrate layer 212.

The dielectric substrate layers 208 may include any dielectric material. For example, the dielectric substrate layers 208 may include one or more of polytetrafluoroethylene (PTFE), ceramic, mica, plastic, a polymer (such as polydimethylsiloxane (PDMS)), or the like.

In some embodiments, the dielectric superstrate layer 214 may include a different dielectric material than the first dielectric substrate layer 210 and the second dielectric substrate layer 212. For example, the dielectric superstrate layer 214 may be biocompatible with the corresponding living tissue and may be designed to resist damage to components of the antenna 106 (such as the second conductive patch 206).

In some embodiments, the first dielectric substrate layer 210 and the second dielectric substrate layer 212 may include a dielectric laminate, such as RO3010™ RO3000™, RO3210™, each available from Rogers Corporation of Rogers, Conn. In some embodiments, the dielectric superstrate layer 214 may include a polymer, such as PDMS, which is relatively biocompatible. In some embodiments, the dielectric superstrate layer 214 may entirely surround the antenna 106 (e.g., may encapsulate all other components of the antenna 106).

In some embodiments, the antenna 106 may include a layer, such as a thin layer, of biocompatible encapsulation that encapsulates the antenna 106 instead of, or in addition to, the dielectric superstrate layer 214.

In some embodiments, the antenna 106 may include an adhesive located between two or more elements and designed to bond the two or more elements together. For example, the adhesive may be located between two or more of the conductive ground plane 200, the first dielectric substrate layer 210, the first conductive patch 204, the second dielectric substrate layer 212, the second conductive patch 206, or the dielectric superstrate layer 214.

The antenna 106 may further include a shorting pin 216. The shorting pin 216 may include a conductive material, such as a metal (copper, gold, or the like) and may extend from the conductive ground plane 200 through the first dielectric substrate layer 210 to the first conductive patch 204. The shorting pin 216 may provide a conductive path from the conductive ground plane 200 to the first conductive patch 204 and may allow for miniaturization of the antenna 106 (i.e., use of the shorting pin 216 may allow a diameter 226 of the antenna 106 and/or an antenna thickness 232 of the antenna 106 to become reduced relative to lack of use of the shorting pin 216).

The conductive ground plane 200, the first conductive patch 204, the first dielectric substrate layer 210, and the second dielectric substrate layer 212 may define a feed point slot 218. The feed point slot 218 may be designed to receive a lead 220. The lead 220 may be electrically connected to one or more of the conductive ground plane 200, the first conductive patch 204, and the second conductive patch 206, and may facilitate transmission of a signal between the antenna 106 and the sensor or stimulator 104 of FIG. 1. In that regard, the lead 220 may excite one or more of the conductive ground plane 200, the first conductive patch 204, and the second conductive patch 206. In some embodiments, the lead 220 may or may not contact the conductive ground plane 200.

In some embodiments, the lead 220 may be an inner coaxial conductor of a coaxial connector 222. The coaxial connector 222 may include an outer coaxial conductor 224 that is electrically connected to the conductive ground plane 200. In that regard, the coaxial connector 222 may function as the physical connector 110 of FIG. 1.

Because the antenna 106 is implantable, it is desirable for a size of the antenna 106 to be relatively small. In that regard, the antenna 106 may have the diameter 226 and the antenna thickness 232. It may be desirable for the diameter 226 and the antenna thickness 232 to be as small as possible. For example, the diameter 226 may be between 2 millimeters (mm) and 20 mm (0.079 inches and 0.79 inches), and the thickness may be between 2 millimeters (mm) and 20 mm (0.079 inches and 0.79 inches).

Each of the vertically stacked conductive patches 202 may include slots. For example, the first conductive patch 204 may include one or more first slot 300, and the second conductive patch 206 may include one or more second slot 302. In the example antenna 106, the first conductive patch 204 includes five slots including a first slot 304 (L1), a second slot 306 (L2), a third slot 308 (L3), a fourth slot 310 (L4), and a fifth slot 312 (L5). The second conductive patch 206 includes six slots including a first slot 314 (L1′), a second slot 316 (L2′), a third slot 318 (L3′), a fourth slot 320 (L4′), a fifth slot 322 (L5′), and a sixth slot 324 (L6′).

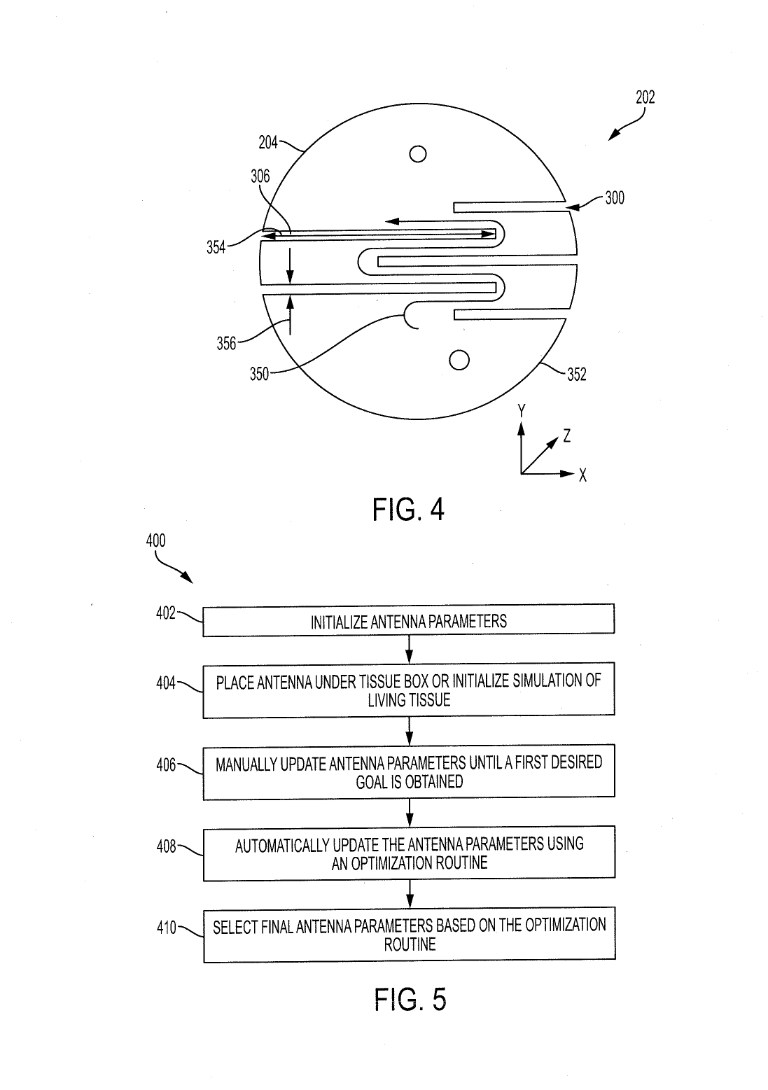

Referring to FIGS. 3 and 4, each of the vertically stacked conductive patches 202 may include a perimeter. For example, the first conductive patch 204 includes a perimeter 352. Each of the slots 300 may extend inward from the perimeter 352. Each of the slots 300 may create a discontinuity along the perimeter 352.

In that regard, the slots 300 may form a serpentine conductive path along the surface of the first conductive patch 204 that is illustrated by an arrow 350. The serpentine conductive path may increase an effective length of a current flow along the first conductive patch 204 (i.e., cause meandering of the current along the first conductive patch 204). Along with increasing the effective length of the current flow, the serpentine conductive path may also reduce antenna resonance frequency for a given physical dimension. The serpentine conductive path may further assist in miniaturization (i.e., the serpentine conductive path may allow the diameter 226 and the antenna thickness 232 to become reduced in size).

Likewise, the slots 302 of the second conductive patch 206 may form a serpentine conductive path along the surface of the second conductive patch 206 that provides the same benefits as the serpentine conductive path of the first conductive patch 204.

The antenna 106 may be designed to operate at a specific frequency. The antenna 106 may be optimized to a specific frequency by adjusting various parameters of the antenna 106. Referring to FIGS. 2, 3, and 4, the parameters may include, for example, a material of each of the conductive ground plane 200, the first conductive patch 204, and the second conductive patch 206. The parameters may also include a total quantity of vertically stacked conductive patches 202. The parameters may also include a shape of each of the conductive ground plane 200, the first conductive patch 204, and the second conductive patch 206 (i.e., one or more of these elements may have any shape including disk shaped (as shown), triangular, rectangular, or the like).

The parameters may also optionally include a material and thickness of any additional encapsulation. The parameters may also optionally include a material and thickness of any adhesive used to bond two or more components of the antenna together.

The parameters may also include a quantity of the slots 300 on the first conductive patch 204 and a quantity of the slots 302 on the second conductive patch 206. The parameters may also include a slot length 354 of each of the slots 300, 302 on the conductive patches 202 (the slot length 354 may be different for each of the slots 300, 302, may be the same for each of the slots 300, 302, or may be the same for some of the slots 300, 302 and different for other slots 300, 302). The parameters may also include a slot width 356 of each of the slots 300, 302 on the conductive patches 202 (the slot width 356 may be different for each of the slots 300, 302, may be the same for each of the slots 300, 302, or may be the same for some of the slots 300, 302 and different for other slots 300, 302).

The parameters may also include a location of the shorting pin 216 and a location of the feed point slot 218. As shown, the location of the feed point slot 218 on the antenna 106 is along an axis 326 (at which the slots 300, 302 run perpendicular), and the location of the shorting pin 216 is offset from the axis 326.

The parameters may also include a thickness 228 of each of the conductive ground plane 200, the first conductive patch 204, and the second conductive patch 206 (the thickness 228 may be the same or different for each of the conductive ground plane 200, the first conductive patch 204, and the second conductive patch 206). The parameters may also include a thickness 230 of each of the dielectric substrate layers 208 (the thickness 230 of each of the first dielectric substrate layer 210, the second dielectric substrate layer 212, and the dielectric superstrate layer 214 may be the same or different). The parameters may also include the diameter 226 of the antenna 106, a diameter of each of the conductive ground plane 200, the first conductive patch 204, the second conductive patch 206, the first dielectric substrate layer 210, the second dielectric substrate layer 212, and the dielectric superstrate layer 214.

Turning now to FIG. 5, a method 400 for optimizing antenna parameters of an implantable or ingestible antenna (such as the antenna 106 of FIG. 1) is shown. The method 400 may be used to optimize one or more of the antenna parameters described above. The antenna parameters may be optimized to achieve a desired effect or goal of the antenna. For example, it may be desirable for the antenna to operate at a desired frequency or within a desired frequency range. In that regard, the desired affected or goal may include one or more of a magnitude of a reflection coefficient at a desired operation frequency, a gain of the antenna at a desired operation frequency, a direct activity of the antenna signal at a desired operation frequency, a pattern of the antenna signal at a desired operation frequency, an efficiency of the antenna at a desired operation frequency, or a bandwidth specification of the antenna.

In block 402, the antenna parameters may be initialized. The antenna parameters may be initialized randomly or may be manually selected based on the desired goal or effect. For example, an antenna designer may be sufficiently knowledgeable to select a quantity of conductive patches, an antenna diameter, a shorting pin location, and a feed point slot location that will provide desirable operation at a specific frequency. The antenna designer may initialize those parameters based on his knowledge, and the remaining parameters may be randomly initialized.

In block 404, the antenna with the initialized parameters may be placed under a tissue box that simulates the living tissue and which the antenna will be used, or a simulation of the antenna may be initialized such that the antenna operation will be simulated within a simulation of the living tissue.

In block 406, the antenna parameters (some or all, some may be different than the antenna parameters manually initialized in block 402) may be manually updated until a first desired goal is obtained. For example, the first desired goal may correspond to a specific reflection coefficient at a desired frequency of operation of the antenna.

Continuing the example, the antenna parameters may be manually updated until the antenna provides a reflection coefficient of negative 15 decibels (dB) at a desired frequency of operation of the antenna. After updating the antenna parameters, the antenna may be tested under the tissue box, or the simulation of the antenna may be performed to measure the operation of the antenna to determine whether the first goal has been achieved. The measuring or simulation of the antenna under the tissue box may be performed after each update of the parameters. After the antenna has been updated to achieve the first desired goal (i.e., parameters have been selected that result in the antenna achieving the first desired goal), the antenna may be considered to be a refined antenna.

In block 408, the antenna parameters may be automatically updated using an optimization routine. The optimization routine may be used or selected to improve upon the first desired goal (e.g., an optimization routine may be used to improve the reflection coefficient at the desired frequency above the desired negative 15 dB), or the optimization routine may be used to improve upon or reach another desired goal (such as to obtain an antenna gain of at least −5 dB). In some embodiments, the optimization routine may be performed a predetermined quantity of times, and the antenna parameters that provide the ideal results may be selected as the final antenna. In some embodiments, the optimization routine may be performed iteratively until a second desired goal is reached (e.g., a reflection coefficient at a desired frequency has reached or exceeded negative 20 dB).

In some embodiments, the optimization routine may be used to obtain multiple goals simultaneously. For example, the optimization routine may be performed to achieve a reflection coefficient above negative 20 dB and a gain of at least 0 dB at the desired frequency.

Some antenna parameters may be set (such as a quantity of conductive patches and a diameter of the conductive patches) and not changed during one or more of blocks 406 and 408, and some antenna parameters (such as a quantity of slots, slot lengths, and slot widths) may be adjustable during one or more of blocks 406 and 408. In some embodiments, some antenna parameters may be adjustable during block 406 and not block 408, and vice versa.

After the optimization routine has been performed in block 408, the final antenna parameters may be selected in block 410. The final antenna parameters may be selected based on the optimized parameters determined in block 408. After the final antenna parameters have been selected, the antenna may be manufactured and connected to the sensor or stimulator. The sensor data may then be transmitted to a remote device via the antenna, or a signal for stimulating the tissue may be received by the antenna. In some embodiments, the antenna may receive a wireless charging signal for wireless charging of the sensor or stimulator.

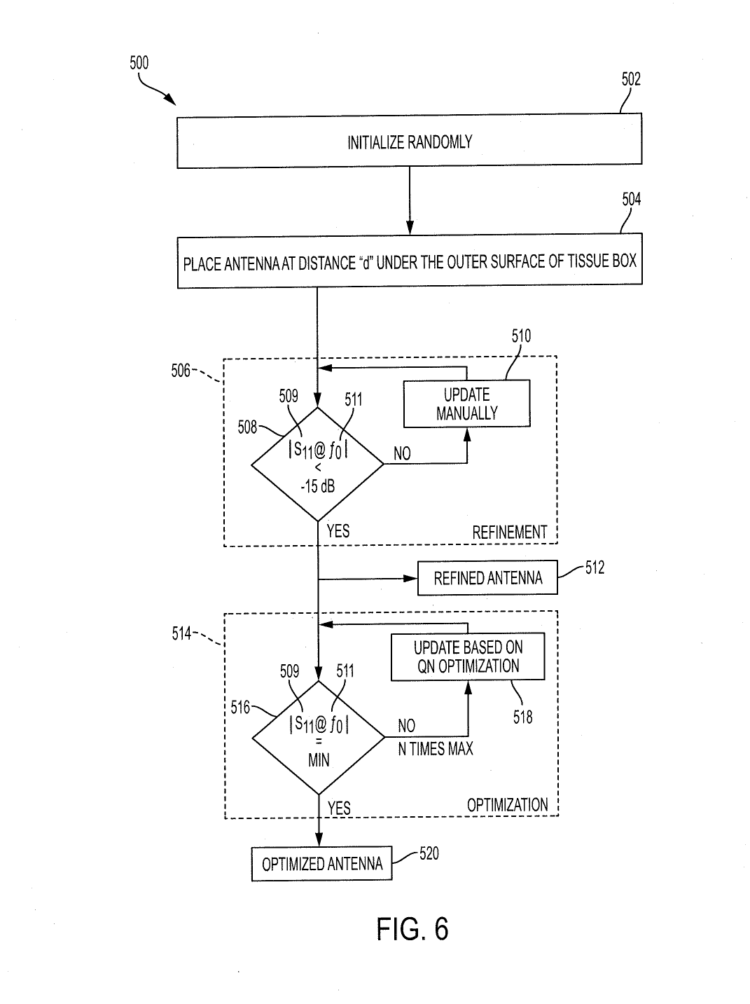

Turning now to FIG. 6, a method 500 illustrates an exemplary implementation of the method 400 of FIG. 4. The method 500 was used to select the antenna parameters of the antenna 106 of FIG. 1. As will be discussed below, desirable results were achieved using the method 500.

In block 502, antenna parameters may be randomly initialized. The antenna parameters may include any one or combination of the antenna parameters described above.

In block 504, the antenna may be positioned (or simulated to be positioned) at a distance “d” beneath an outer surface of a tissue box. The distance “d” may correspond to actual air to antenna separation distance for the desired medical application (and may correspond to implantation depth).

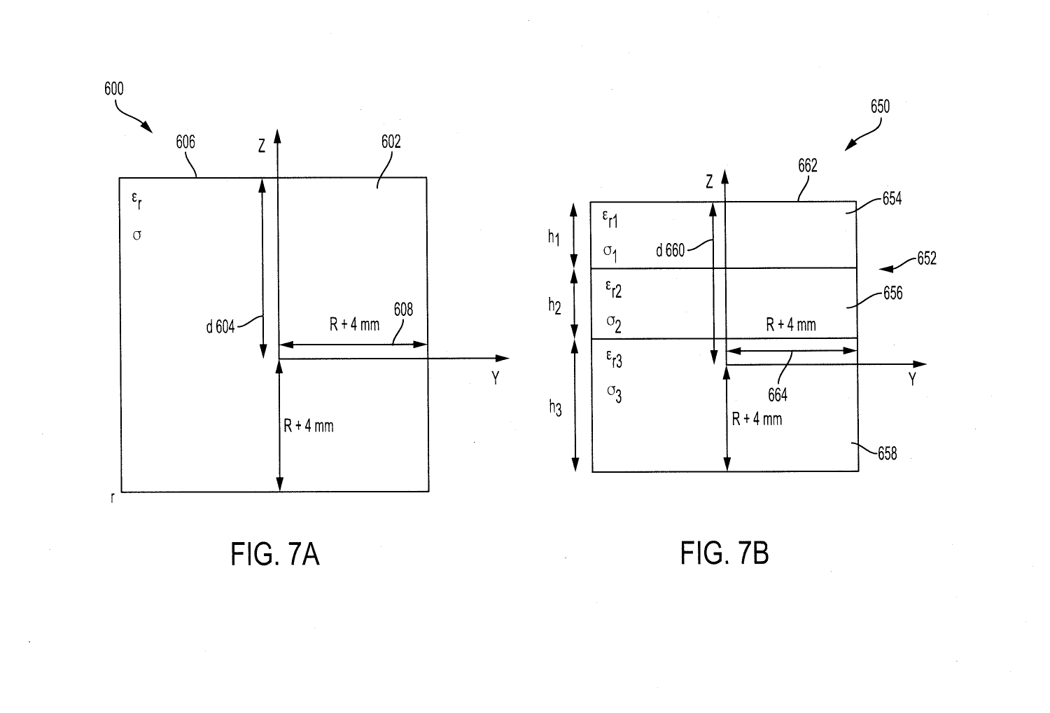

For example and referring to FIG. 7A, an example tissue box 600 is shown. The tissue box 600 may be a single layer tissue model (SLTM) meaning that the tissue box 600 includes a single tissue 602. The single tissue 602 may simulate, for example, the dielectric properties (relative permittivity (εᵣ) and conductivity (σ)) of the intended implantation tissue. The tissue may include skin, muscle, or the like. The antenna (or model of the antenna) may be positioned at the distance “d” 604 beneath a top surface 606 of the tissue box 600.

The tissue box 600 may have a radius 608 that is greater than a radius of the corresponding antenna. For example, the radius 608 may be equal to the radius (i.e., half of the diameter 226 of the antenna 106 of FIG. 2) of the corresponding antenna plus an additional 4 millimeters (4 mm) in the X and Y directions.

As another example and referring to FIG. 7B, another example tissue box 650 is shown. The tissue box 650 may be a multiple layer tissue model (MLTM) meaning that the tissue box 650 simulates the dielectric properties (relative permittivity (εᵣ) and conductivity (σ)) of multiple tissues 652. The multiple tissues 652 include skin 654 (having a thickness of 5 mm), bone 656 (having a thickness of 5 mm), and brain 658 (having a thickness of 3 mm). The antenna (or model of the antenna) may be positioned at the distance “d” 660 beneath a top surface 662 of the tissue box 650. The tissue box 650 may have a radius 664 that is greater than a radius of the corresponding antenna. For example, the radius 664 may be equal to the radius of the corresponding antenna plus an additional 4 mm.

Referring to FIGS. 7A and 7B, the size of the tissue boxes 600, 650 was selected for antenna operational frequencies in the MedRadio band (i.e., the Medical Device Radiocommunications Service, a specification created by the United States Federal Communications Commission) such as between 401 MHz to 406 MHz. It was discovered that further enlargement of the tissue boxes 600, 650 provided minimal improved results. However, different size tissue boxes may be desirable for other operational frequencies.

It is expected that the tissue box 650 (the MLTM tissue box) may provide greater accuracy as it accounts for the dielectric loading effect of multiple tissues. On the other hand, the tissue box 600 (the SLTM tissue box) is easier to model and can be meshed more coarsely, which may render the SLTM tissue box variation slightly faster to implement.

Returning reference to FIG. 6 and in block 506, a refinement procedure may be performed to refine the antenna parameters of the antenna. The refinement procedure may be performed manually rather than automatically.

In particular and in block 508, it may be determined whether the reflection coefficient 509 of the antenna signal at the desired frequency 511 is less than (or equal to) negative 15 dB. This may be determined by testing a physical representation of the antenna in the tissue box and measuring the reflection coefficient 509 at the desired frequency 511, or by running a simulation of the antenna in the tissue box and calculating the reflection coefficient 509 at the desired frequency 511.

If the reflection coefficient 509 at the desired frequency 511 is greater than (or equal to) negative 15 dB then the method 500 may proceed to block 510 where the antenna parameters may be updated manually. The goal of negative 15 dB was selected so as to provide a relatively safe margin for the reflection coefficient performance. This goal may be modified per design requirements or designer preference. For example, a designer of the antenna may update one or more of the antenna parameters, such as a quantity of slots on each of the conductive patches, a length of one or more of the slots, a width of one or more of the slots, or the like.

If the reflection coefficient 509 at the desired frequency 511 is less than negative 15 dB then the refinement procedure of block 506 may be complete, and the antenna with the updated antenna parameters may be referred to as a refined antenna 512.

After the refined antenna 512 has been created during the refinement procedure in block 506, the method 500 may proceed to an optimization procedure in block 514. The optimization procedure in block 514 may be used to optimize performance (i.e., optimize the desired goal or achieve one or more secondary goal). For example and in block 516, it may be determined whether the reflection coefficient 509 at the desired frequency 511 is equal to a minimum reflection coefficient 509. This may be determined by testing a physical representation of the antenna in the tissue box and measuring the reflection coefficient 509 at the desired frequency 511, or by running a simulation of the antenna in the tissue box and calculating the reflection coefficient 509 at the desired frequency 511.

After block 516 has been performed, the method may proceed to block 518 where an optimization algorithm updates the antenna parameters. For example, the optimization algorithm may include a Quasi-Newton (QN) optimization method. In some embodiments, the optimization algorithm performed in block 518 may include any other optimization algorithm that is known in the art. For example, the optimization algorithm may include an iterative method (such as Newton's method, sequential quadratic programming, an ellipsoid method, a gradient descent method, the QN method or the like), a convergence algorithm, a heuristic algorithm (such as a memetic algorithm, differential evolution, dynamic relaxation, or the like), or any other optimization algorithm.

As shown in FIG. 6, the QA method is used in block 518 as the optimization algorithm to update the antenna parameters. In that regard, the optimization procedure in block 514 may be performed “N” number of times, or may cease performing when a second goal is achieved (such as the reflection coefficient reaching a minimum value). “N” may be selected based on a trade-off between an amount of time available to perform optimization and a desire for highest quality optimization of the antenna parameters. After the optimization algorithm in block 518 has been performed “N” quantity of times (or the second goal is achieved), the set of antenna parameters having the lowest reflection coefficient 509 at the desired frequency 511 may be set as the finalized antenna parameters. The resulting antenna having the finalized antenna parameters may be referred to as an optimized antenna 520.

Referring now to FIGS. 3, 6, 7A, 8, 10, and 11, the method 500 was used twice to optimize parameters of the antenna 106. In particular, the method 500 was used to optimize a reflection coefficient of the antenna 106 at 403 Megahertz (MHz) for intra-cranial pressure (ICP) monitoring applications.

MedRadio operation inside the skin tissue of the human head was considered. Finite element (FE) simulations were carried out using a high frequency electromagnetic field simulation software (Ansys HFSS, available from Ansys of Canonsburg, Pa.). The FE solver automatically meshed the geometry in an automated way. The mesh was perturbed by 30% between each pass, and the meshing procedure stopped when the maximum change in the magnitude of the reflection coefficient between two consecutive passes was less than 0.02 or when the number of passes exceeded 10. A Quasi-Newton optimizer was integrated into the platform. The radius of the conductive ground plane 200 was assumed to be 6 mm, while the radius of the each of the conductive patches 204, 206 was assumed to be 5 mm. Both dielectric substrates 210, 212 and the dielectric superstrate layer 214 were considered to be 0.6 mm-thick, and were made out of RO3210™ material. Five and six slots were considered upon the lower and upper patches, respectively. Simulations were performed using a personal computer (PC) having a 2.83 Gigahertz (GHz) processor and 3.25 Gigabytes (GB) of installed random access memory (RAM).

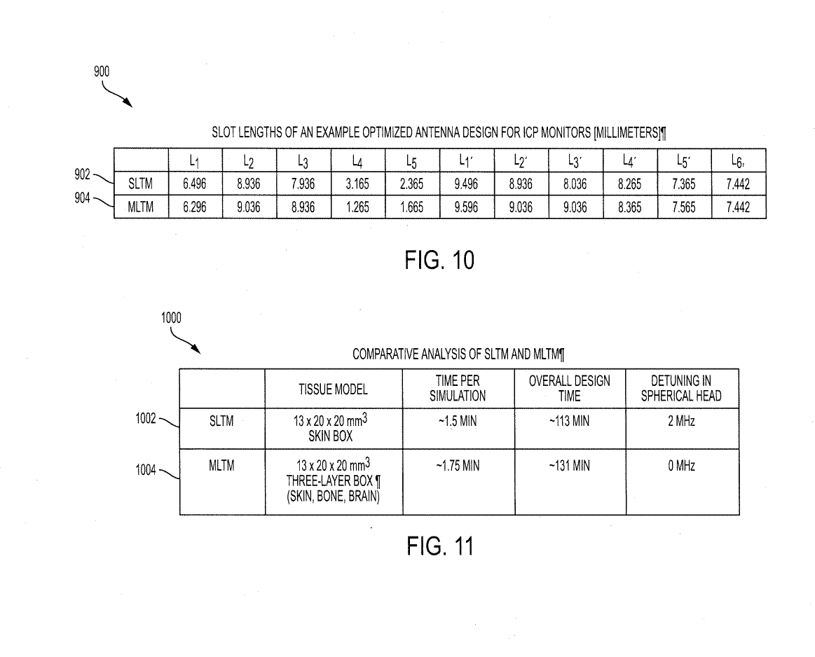

The tissue box 600 that includes the single tissue 602 was selected to be used in the simulations of blocks 508 and 516 during the first iteration of the method 500. The optimization procedure in block 514 was performed 75 times (i.e., “N” was equal to 75). As shown in a first row 1002 of a chart 1000 of FIG. 11, simulations took approximately 1.5 minutes each to perform with a total simulation time of 113 minutes. The optimized antenna 106, 520 includes the first conductive patch 204 having five slots 300, and the second conductive patch 206 having six slots 302. The slot length of each of the slots 300, 302 is shown in a first row 902 of a chart 900 illustrated in FIG. 10 and measured in millimeters.

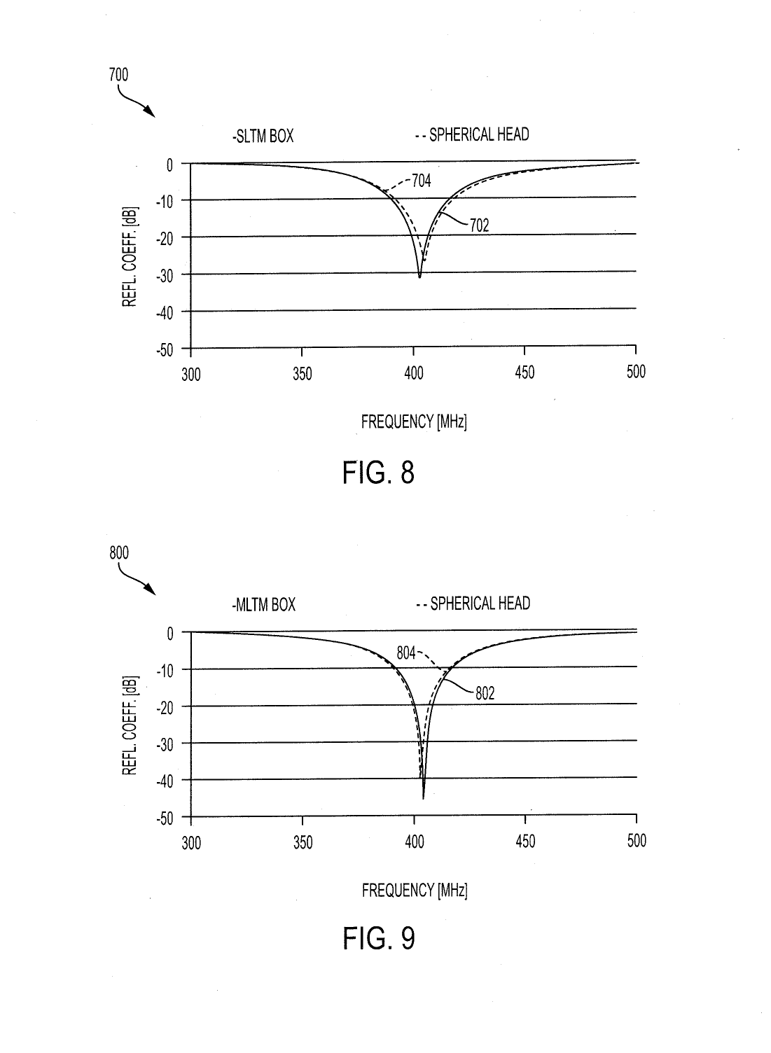

As shown in a graph 700 of FIG. 8, a measured reflection coefficient 704 using the optimized antenna 106, 520 is relatively close to a desired performance 702 of the antenna. As shown in the first row 1002 of the chart 1000, the measured reflection coefficient 704 was detuned from the desired performance 702 by only 2 MHz, which corresponds to relatively desirable operation of the optimized antenna 106, 520.

Referring now to FIGS. 3, 6, 7B, 9, 10, and 11, the second iteration of the method 500 was used to optimize parameters of the antenna 106 using the tissue box 650 (the MLTM tissue box). In particular, the method 500 was used to optimize a reflection coefficient of the antenna 106 at 403 Megahertz (MHz).

During this iteration of the method 500, the tissue box 650 that includes the multiple tissues 652 was selected to be used in the simulations of blocks 508 and 516. Again, the optimization procedure in block 514 was performed 75 times (i.e., “N” was equal to 75). As shown in a second row 1004 of the chart 1000 of FIG. 11, simulations took approximately 1.75 minutes each to perform with a total simulation time of 131 minutes. Thus, simulation with the tissue box 650 that includes the multiple tissues 652 took approximately 18 minutes longer than simulation using the SLTM. The optimized antenna 106, 520 includes the first conductive patch 204 having five slots 300, and the second conductive patch 206 having six slots 302. The slot length of each of the slots 300, 302 is shown in a second row 904 of the chart 900 illustrated in FIG. 10 and measured in millimeters.

As shown in a graph 800 of FIG. 9, a measured reflection coefficient 804 using the optimized antenna 106, 520 is relatively close to a desired performance 802 of the antenna. As shown in the second row 1004 of the chart 1000, the measured reflection coefficient 704 was detuned from the desired performance 702 by 0 MHz, which is desirable and corresponds to nearly perfect performance of the optimized antenna 106, 520.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents. Where used throughout the disclosure and claims, “at least one of A or B” includes “A” only, “B” only, or “A and B.”

What is claimed is:

1. An antenna for use in living tissue and designed to at least one of receive or transmit signals, the antenna comprising:
a conductive ground plane;
a first conductive patch having at least a first slot;
a second conductive patch having at least a second slot, the conductive ground plane, the first conductive patch, and the second conductive patch being stacked;
a first dielectric substrate layer located between the conductive ground plane and the first conductive patch; and
a second dielectric substrate layer located between the first conductive patch and the second conductive patch.

2. The antenna of claim 1 wherein the at least the first slot includes at least two slots that create a serpentine conductive path along the first conductive patch to increase an effective length of a current flow along the first conductive patch.

3. The antenna of claim 1 wherein the first conductive patch has a perimeter and the at least one the first slot extends inward from the perimeter and creates a non-conductive discontinuity at the perimeter.

4. The antenna of claim 1 wherein each of the conductive ground plane, the first conductive patch, the first dielectric substrate layer, and the second dielectric substrate layer define a feed point slot for receiving a lead configured to be connected to a sensor or a stimulator implanted with the antenna and to each of the conductive ground plane, the first conductive patch, and the second conductive patch.

5. The antenna of claim 4 further comprising a coaxial connector having the lead as an inner coaxial conductor and an outer coaxial conductor that is electrically coupled to the conductive ground plane.

6. The antenna of claim 5 further comprising at least one of the sensor coupled to the antenna via the coaxial connector and configured to detect data corresponding to the living tissue, or the stimulator coupled to the antenna via the coaxial connector and configured to stimulate the living tissue.

7. The antenna of claim 1 further comprising a shorting pin extending through the first dielectric substrate layer and electrically connected to the conductive ground plane and the first conductive patch.

8. The antenna of claim 1 further comprising a dielectric superstrate layer in contact with the second conductive patch such that the second conductive patch is sandwiched between the dielectric superstrate layer and the second dielectric substrate layer, the dielectric superstrate layer configured to be biocompatible with the living tissue and to resist damage to the second conductive patch by the living tissue.

9. The antenna of claim 1 wherein each of the conductive ground plane, the first conductive patch, the second conductive patch, the first dielectric substrate layer, and the second dielectric substrate layer are disk shaped.

10. An implantable or ingestible device for physiological monitoring or stimulation of tissue, comprising:
an antenna configured to be connected to a sensor or a stimulator for wirelessly sending and receiving monitoring or stimulation data of the tissue, the antenna having:
a conductive ground plane,
at least two vertically stacked conductive patches including a lower conductive patch and an upper conductive patch, the at least two vertically stacked conductive patches configured to at least partially provide the monitoring or stimulation data to at least one of the sensor, the stimulator, or a remote device, and having one or more slots that assist in miniaturization, and
one or more dielectric substrate layers that insulate the conductive ground plane from the lower conductive patch, the lower conductive patch from the upper conductive patch, and the upper conductive patch from the tissue.

11. The implantable or ingestible device of claim 10 further comprising a shorting pin that shorts the lower conductive patch to the conductive ground plane to further assist in miniaturization.

12. The implantable or ingestible device of claim 10 wherein the one or more slots create a serpentine shape in each of the at least two vertically stacked conductive patches to increase an effective length of a current flow.

13. The implantable or ingestible device of claim 10 further comprising a coaxial cable that connects to the conductive ground plane and excites both the lower conductive patch and the upper conductive patch.

14. The implantable or ingestible device of claim 13 wherein the coaxial cable includes an inner coaxial conductor that contacts the conductive ground plane, the lower conductive patch, and the upper conductive patch and transmits a signal between the antenna and the sensor or the stimulator.

15. A method of optimizing antenna parameters of an implantable or ingestible antenna, comprising:
randomly or manually initializing each antenna parameter of a plurality of antenna parameters;
manually updating at least some of the plurality of antenna parameters until a first desired goal corresponding to antenna behavior is obtained;
automatically updating, using an optimization routine, at least some of the plurality of antenna parameters a predetermined quantity of iterations; and
selecting, based on optimized antenna behavior, a plurality of final antenna parameters.

16. The method of claim 15 wherein automatically updating at least some of the plurality of antenna parameters further includes simulating, using a tissue-simulating model, the antenna behavior in a simulation of living tissue.

17. The method of claim 16 wherein simulating, using the tissue-simulating model, the antenna behavior includes simulating the antenna behavior in a model that includes multiple types of tissues.

18. The method of claim 15 wherein the antenna behavior includes at least one of a magnitude of a reflection coefficient at a desired operation frequency, a gain of the antenna at the desired operation frequency, a direct activity of an antenna signal of the antenna at the desired operation frequency, a pattern of the antenna signal at the desired operation frequency, an efficiency of the antenna at the desired operation frequency, or a bandwidth specification of the antenna.

19. The method of claim 15 wherein the antenna includes:
a conductive ground plane;
a first conductive patch having at least a first slot;
a second conductive patch having at least a second slot, the conductive ground plane, the first conductive patch, and the second conductive patch being stacked;
a first dielectric substrate layer located between the conductive ground plane and the first conductive patch;
a second dielectric substrate layer located between the first conductive patch and the second conductive patch.

20. The method of claim 19 wherein the plurality of antenna parameters includes at least one of:
a material of each of the first conductive patch, the second conductive patch, and the conductive ground plane;
a shape of each of the first conductive patch, the second conductive patch, and the conductive ground plane;
a total quantity of patches;
a quantity of each of first slots of the first conductive patch and second slots of the second conductive patch;
a length of each of the first slots and of each of the second slots;
a width of each of the first slots and of each of the second slots;
a location of a short pin of the antenna;
a material of the short pin of the antenna;
a location of a feed point slot of the antenna;
a material of a lead extending through the feed point slot of the antenna;
a thickness of each of the first dielectric substrate layer and the second dielectric substrate layer;
a thickness of a superstrate layer of the antenna;
a thickness of each of the first conductive patch, the second conductive patch, and the conductive ground plane; and
a diameter of the antenna.
